(19) United States
(12) Patent Application Publication
(10) Pub. No.: US 2025/0311935 A1
(43) Pub. Date: Oct. 9, 2025

(54) MEDICAL SYSTEM AND METHOD WITH A MOBILE DEVICE TO MIRROR PROMPTS IN A SECOND LANGUAGE

(71) Applicant: West Affum Holdings DAC, Dublin (IE)

(72) Inventors: Brian D. Webster, Mercer Island, WA (US); Jeffrey M. Boschee, Woodinville, WA (US); Phillip D. Foshee, JR., Woodinville, WA (US); Quan H. Nguyen, Renton, WA (US); Bryce L. Greenhalgh, Woodinville, WA (US)

(73) Assignee: West Affum Holdings DAC, Dublin (IE)

(21) Appl. No.: 19/169,604

(22) Filed: Apr. 3, 2025

Related U.S. Application Data
(60) Provisional application No. 63/575,507, filed on Apr. 5, 2024.

(51) Int. Cl. A61B 5/024; A61B 5/00

(52) U.S. Cl. CPC A61B 5/02405; A61B 5/0022; A61B 5/4836; A61B 5/6802; A61B 5/7405; A61B 5/742; A61B 2560/0276

(57) ABSTRACT

A medical system, a method, and a non-transitory computer readable medium for outputting an audible alert in a second language are disclosed. The medical system includes a Wearable Medical Device (WMD) including at least one medical device speaker. The WMD is configured to provide therapy to a patient upon detection of a cardiac condition of the patient based on patient data. The medical system further includes a mobile device including at least one processor and at least one mobile device speaker. The mobile device is in communication with the WMD. The at least one processor of the mobile device is configured to receive an indication of an audible alert to be output by the at least one medical device speaker of the WMD in a first language, and cause the at least one mobile device speaker to output the audible alert in a second language.

### CROSS-REFERENCE TO RELATED APPLICATIONS

This application claims priority to and the benefit of the provisional patent application No. 63/575,507 titled “WCD SYSTEM WITH ASSISTANT DEVICE TO MIRROR PROMPTS IN A SECOND LANGUAGE,” filed in the United States Patent and Trademark Office on Apr. 5, 2024. The specification of the above referenced patent application is incorporated herein by reference in its entirety.

### TECHNICAL FIELD

The present technology relates to the field of Wearable Medical Devices (WMDs) and more particularly, but not by way of limiting, the present technology relates to a medical system that allows audible alerts generated by a WMD to be output in a user-preferred language via a mobile device, based on communication between the WMD and the mobile device. The medical system facilitates the mirroring of the audible alerts to accommodate the user-preferred language.

### BACKGROUND

Wearable Medical Devices (WMDs) are portable, user-friendly health monitoring devices designed to be worn on a body, allowing for continuous tracking of various physiological parameters. By offering continuous monitoring, the WMDs ensure that wearers (e.g., patients) manage chronic conditions, track recovery progress, and detect potential health issues early.

Specifically, the WMD monitors whether the patients are at risk of Sudden Cardiac Arrest (SCA) due to life-threatening arrhythmias. If the WMD detects an abnormal heart rhythm, such as a life-threatening arrhythmia, the WMD delivers a life-saving shock. The WCD is equipped with sensors that track heart's electrical activity, ensuring prompt action in an event of cardiac emergency. In addition to providing continuous monitoring, the WMD often issues alerts or prompts to notify the patients of impending shocks or other critical events, enabling the patients to respond appropriately to potentially life-threatening situations. A significant issue arises when the patients do not respond adequately to such alerts or prompts due to language barriers or preferences.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure relates to a medical system for outputting an audible alert in a second language. In an aspect of the present disclosure, the medical system includes a Wearable Medical Device (WMD) including at least one medical device speaker. The WMD is configured to provide therapy to a patient upon detection of a cardiac condition of the patient based on patient data. The medical system further includes a mobile device including at least one processor and at least one mobile device speaker. The mobile device is in communication with the WMD. The at least one processor is configured to receive an indication of an audible alert to be output by the at least one medical device speaker of the WMD in a first language, and cause the at least one mobile device speaker to output the audible alert in a second language.

In some examples, the indication is the audible alert output by the at least one medical device speaker.

In some examples, the indication is a pre-alert signal.

In some examples, the pre-alert signal is output by the at least one medical device speaker, the pre-alert signal having a frequency and/or amplitude that is inaudible to humans.

In some examples, the pre-alert signal is an electronic communication transmitted by the WMD to the at least one processor of the mobile device.

In some examples, the audible alert includes an alert associated with WMD conditions and/or associated with patient data.

In some examples, the at least one processor is further configured to send a suppression communication to the WMD, in response to receiving the pre-alert signal. The suppression communication is operable to override the output of the audible alert in the first language with the audible alert in the second language.

In some examples, the at least one processor is further configured to display a visual alert in the second language corresponding to the audible alert on a display of the mobile device.

In some examples, the at least one processor is further configured to detect a language of a bystander, and cause the at least one mobile device speaker to output the audible alert in the detected language.

The present disclosure further describes a method for implementing the medical system provided herein. The present disclosure also describes computer-readable media coupled to one or more processors and having instructions stored thereon which, when executed by the one or more processors, cause the one or more processors to perform operations in accordance with the medical system described herein.

It is appreciated that the medical system in accordance with the present disclosure can include any combination of the aspects and features described herein. That is, the medical system in accordance with the present disclosure is not limited to the combinations of aspects and features specifically described herein, but also include any combination of the aspects and features provided.

The details of one or more implementations of the present disclosure are set forth in the accompanying drawings and the description below. Other features and advantages of the present disclosure will be apparent from the description and drawings, and from the claims

### BRIEF DESCRIPTION OF THE DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

FIG. 1 illustrates a medical system for outputting an audible alert in a second language, according to an embodiment of the present disclosure.

FIG. 2 illustrates an example architecture of a language mirroring engine coupled to a database, within a server disclosed in FIG. 1, according to an embodiment of the present disclosure.

FIG. 3 illustrates an example scenario of outputting an audible alert in a second language upon recognition of the audible alert in a first language, according to an embodiment of the present disclosure.

FIG. 4 illustrates an example scenario of suppressing an audible alert in a first language and outputting the audible alert in a second language, according to an embodiment of the present disclosure.

FIG. 5 illustrates an example scenario of outputting an audible alert in a second language in response to receiving a pre-alert signal from a Wearable Medical Device (WMD), according to an embodiment of the present disclosure.

FIG. 6 illustrates an example scenario of outputting an audible alert in a second language upon detecting an audio output state of a WMD, according to another embodiment of the present disclosure.

FIG. 7 illustrates an example method for outputting an audible alert in a second language, according to an embodiment of the present disclosure.

FIG. 8 illustrates an example method for overriding an audible alert in a first language with the audible alert in a second language, according to an embodiment of the present disclosure.

FIG. 9 illustrates an example User Interface (UI) of a mobile device displaying language settings of the mobile device, according to an embodiment of the present disclosure.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures or methods associated with the wearable medical system have not been shown or described in detail to avoid unnecessarily obscuring descriptions of the embodiments.

Unless the context indicates otherwise, throughout the specification and claims which follow, the word “comprise” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context clearly dictates otherwise.

Reference throughout this specification to “one aspect” or “an aspect” means that a particular feature, structure, or characteristic described in connection with an embodiment is included in at least one aspect. Thus, the appearances of the phrases “in one aspect” or “in an aspect” in various places throughout this specification are not necessarily all referring to the same embodiment. Furthermore, the particular features, structures, or characteristics may be combined in any suitable manner in one or more aspects.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Certain terms and phrases have been used throughout the disclosure and will have the following meanings in the context of the ongoing disclosure:

The term “Wearable Medical Devices (WMD)” refers to a medical device worn by a patient, which is designed to monitor, detect, or provide therapeutic intervention for specific medical conditions. In the context of the present disclosure, the WMD is configured to detect cardiac conditions of the patient based on patient data, and detect device-related conditions, such as low battery, loose connection, and/or the like. The WMD may provide necessary therapy to the patient upon detecting the cardiac conditions. The therapy may include but is not limited to providing electrical stimulation or pacing in the form of a shock. The WMD works in conjunction with a mobile device to ensure continuous patient monitoring and efficient data exchange.

The term “mobile device” refers to a portable, handheld electronic device that enables communication of a user or patient with the WMD and/or a server. The mobile device includes hardware components such as a processor, memory, and a User Interface (UI) including an input unit, a display, and a mobile device speaker to execute one or more functions.

The term “voice prompt” refers to an audible alert issued by the WMD, which is used to convey important information regarding a health status of the patient (e.g., arrhythmia detection) or operational condition(s) of the WMD (e.g., a low battery condition, a loose connection, a malfunction, and/or the like).

The term “pre-alert signal” refers to an inaudible or high-frequency signal sent by the WMD prior to issuing the voice prompt. The pre-alert signal acts as a trigger for the mobile device to prepare and potentially suppress issuance of the voice prompt in a default language.

The term “first language” refers to a default language in which the WMD issues the voice prompt. The first language is pre-configured in the WMD and is used to communicate alerts or instructions related to the health status of the patient or the operational condition of the WMD.

The term “second language” refers to a language in which the voice prompt from the WMD in the first language is mirrored or translated by the mobile device. The second language is selected by the patient or automatically determined based on a language setting of the mobile device.

WMDs are designed to monitor patients at risk of SCA and deliver life-saving shocks when necessary. The WMDs may measure a wide range of health metrics, such as a heart rate, a blood pressure, glucose levels, and physical activity, providing real-time data to both the wearer and healthcare providers.

Typically, the WMDs like WCDs are particularly beneficial for patients who are not candidates for Implantable Cardioverter-Defibrillators (ICDs) or those requiring temporary protection until their condition (e.g., cardiac instability) stabilizes. The WCDs are commonly worn by patients (e.g., like a vest or garment) with certain heart conditions, providing continuous monitoring and emergency interventions to prevent fatal arrhythmia. To ensure that the patients are aware of critical events, such as an impending shock or other system conditions, the WCDs issue audible alerts (e.g., voice prompts), prompting the patients to take specific actions. The audible alerts serve as vital communication, helping the patients to understand a particular situation and respond appropriately. However, a challenge arises in cases where the patient does not fully comprehend the audible alerts, either due to a language barrier or a preference for a different voice tone or format.

One of the primary challenges with existing medical systems or the WCDs is that the audible alerts or prompts are typically delivered in a configuration that includes a standard language or with a standard voice tone or format. Such configuration is problematic for patients who do not speak the language in which the audible alerts are issued, or for those who may struggle to understand the standard voice tone or clarity of the voice. For example, a Spanish-speaking patient may find difficulty in comprehending an English voice prompt, potentially resulting in delayed or insufficient responses in critical situations. Furthermore, some patients may have preferences regarding the voice tone, which, if not addressed, may reduce effectiveness of communication between the WCD and the patient. Lack of customization and adaptability in the existing medical systems or the WCDs leads to reduced patient compliance and understanding, which may undermine the effectiveness of the WCD and jeopardize patient safety.

Additionally, in some cases, the patients may not respond to the audible alerts due to timing of prompt delivery or voice format. For example, if the WCD issues an audible alert but the patient is distracted or unable to clearly hear the issued audible alert, then the patient might miss the audible alert, potentially leading to serious health risks. In such cases, the existing medical systems or the WCDs offer limited options for tailoring the audible alerts to improve patient understanding or provide redundancy in communication. Therefore, there is a need for a medical system that is capable of addressing the aforementioned challenges of communication barriers and improving patient compliance.

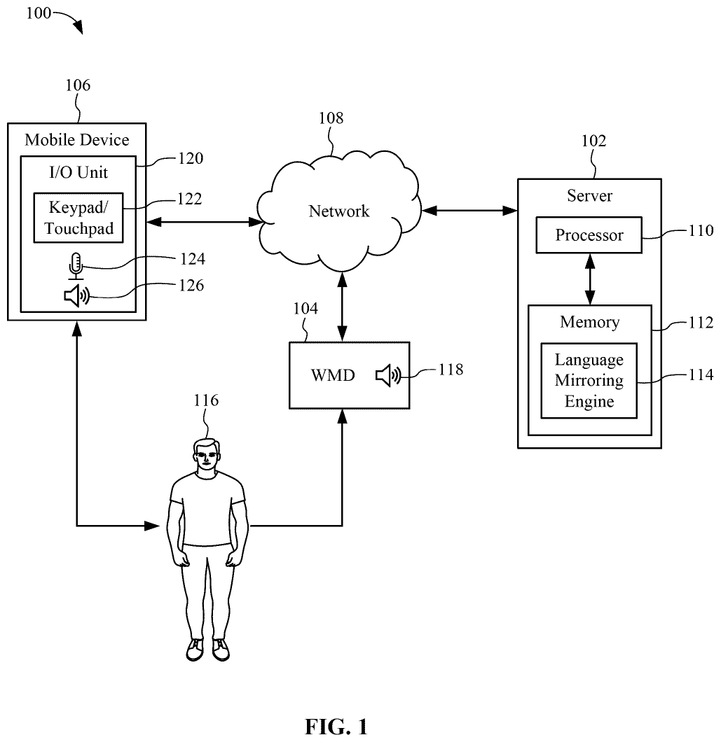

The present disclosure addresses the above discussed challenges by mirroring the audible alerts (generated in a standard language or with a standard voice tone by a WMD or WCD) in a different language, or a voice tone or format, which will be explained further in detail in conjunction with FIGS. 1-9. The present disclosure provides translation of the audible alerts which are in the standard language or with the standard voice tone or format into a language preferred by the patient, such as Spanish, or change voice characteristics (e.g., gender, tone, or the like) to match the preferences of the patient. The translation ensures that the patient receives significant information in a form the patient may understand and respond to more effectively. Additionally, the present disclosure provides suppression or delay in generation of the audible alerts or the voice prompts in the standard language, voice, tone, or format while mirroring, ensuring that the audible alerts or the voice prompts are rendered in the patient's preferred language or voice characteristics, to prevent confusion or overlapping prompts. The disclosure offers a solution that enhances clarity, comprehension, and overall effectiveness of audible alerts, resulting in improved patient compliance and safety. Although the embodiments in the present disclosure are described with respect to the user or patient, they are also applicable to other users, such as bystanders, caregivers, and the like, without departing from the scope of the present disclosure.

FIG. 1 illustrates an example of a medical system 100 for outputting an audible alert in a second language, according to an embodiment of the present disclosure. As depicted in FIG. 1, the medical system 100 may include a server 102, a Wearable Medical Device (WMD) 104, a mobile device 106, and a network 108. For brevity, only one server, mobile device, and WMD are depicted in FIG. 1. However, in some implementations, the medical system 100 may include multiple servers, mobile devices, and/or WMDs without departing from the scope of the present disclosure. The multiple servers may be at the same geographical location or at different geographical locations operating and coordinating in a distributed manner to achieve server functionality, without any limitation.

In some examples, the server 102 may be implemented as an on-premises system that is operated by an organization or a third-party engaged in cross-platform interactions and data management. In some examples, the server 102 may be implemented as an off-premises system (for example, a cloud or an on-demand system) that is operated by an organization or a third-party on behalf of the organization. In some examples, the server 102 may be implemented in a cloud environment. For simplicity, the server 102 depicted in FIG. 1 may be a cloud environment that is intended to represent various forms of servers including a web server, an application server, a proxy server, a network server, a server pool, and/or the like. In some examples, the server 102 may include, but is not limited to, a back-end system, a desktop, a laptop, a notebook, a tablet, a smartphone, a mobile phone, and/or the like.

In an exemplary embodiment, the server 102 may be a customer relation management (CRM) system such as the Salesforce CRM platform (Salesforce) available from Salesforce, Inc., San Francisco, CA, and/or a server provided by the WMD provider such as Kestra CareStation remote data platform. While the following description references Salesforce and the Kestra CareStation platform, the server 102 is not limited to Salesforce and/or the Kestra CareStation platform and may be implemented using one or more other platforms including server(s) and/or cloud-based services provided by the WMD provider.

The server 102 may include at least a processor 110 and a memory 112. In some implementations, the server 102 may include more than one processor. The processor 110 may include, for example, microprocessors, microcomputers, microcontrollers, digital signal processors, central processing units, state machines, logic circuits, and/or any devices that manipulate data or signals based on operational instructions. The memory 112 may be a nonvolatile memory or a volatile memory. Examples of the non-volatile memory may include, but are not limited to, a flash memory, a Read Only Memory (ROM), a Programmable ROM (PROM), Erasable PROM (EPROM), and Electrically EPROM (EEPROM) memory. Examples of the volatile memory may include, but are not limited, a Dynamic Random Access Memory (DRAM), a Static Random-Access Memory (SRAM), and the like.

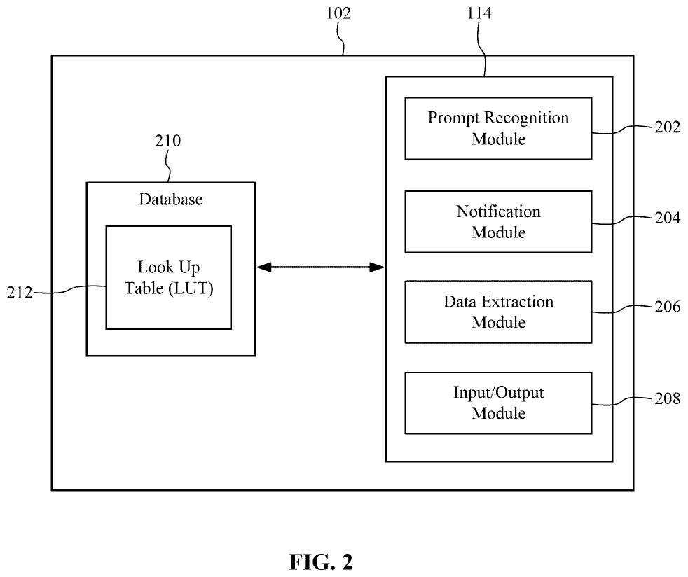

The memory 112 may be communicatively coupled to the processor 110. The memory 112 may store multiple modules or engines including a language mirroring engine 114. In an implementation, the language mirroring engine 114 may be stored as a downloadable application or a library including various instructions. In another implementation, the application may include the language mirroring engine 114. The language mirroring engine 114 may be executed by the processor 110 for outputting the audible alert(s) in the second language. The language mirroring engine 114 may include various operations which are described further in detail in conjunction with FIG. 2 of the present disclosure. The memory 112 may also store various data (e.g., audible alerts, intermediate results generated by modules of the language mirroring engine 114, a Look Up Table (LUT), and/or the like) that may be captured, processed, and/or required by the server 102.

The server 102 may interact with the WMD 104 and/or the mobile device 106 via the network 108. The network 108 may correspond to a communication network. Examples of the network 108 may include, but are not limited to, a Local Area Network (LAN), a Wide Area Network (WAN), the Internet, Wi-Fi, Long Term Evolution (LTE), Intranet, Worldwide Interoperability for Microwave Access (WiMAX), General Packet Radio Services (GPRS), Bluetooth, or a combination thereof. In some examples, the network 108 may be accessed over a wired and/or a wireless communication link.

The WMD 104 may be worn by a user 116. The non-limiting examples of the user 116 may include a patient, a physician, a medical staff member, a wearer, a support staff, a caregiver, a bystander, a family member, a medical professional, a healthcare provider, or the like. Additionally, the user 116 may be responsible for ensuring proper placement and maintenance of the WMD 104, which may include ensuring that the WMD 104 is securely affixed to the body and functioning correctly.

The WMD 104 may monitor the cardiac condition of the user 116. In particular, the WMD 104 may detect various conditions associated with the user 116 such as arrhythmia (e.g., atrial fibrillation or ventricular tachycardia), bradycardia, and/or other abnormal heart rhythms. Also, the WMD 104 may detect other physiological events such as oxygen desaturation, elevated heart rate, and/or increased blood pressure. Additionally, the WMD 104 may be capable of detecting device-related conditions, such as low battery states, device malfunction, sensor disconnection, and/or poor contact with skin of the user 116.

Further, examples of the WMD 104 may include, but are not limited to, a Wearable Cardioverter-Defibrillator (WCD), a Wearable Electrocardiogram (ECG), a wearable glucose monitor, a blood pressure monitor, and/or any WMD capable of detecting physiological conditions. The WMD 104 may include at least one medical device speaker 118. The medical device speaker 118 may output the audible alert in a first language. For example, once a condition associated with the user 116 and/or the device-related condition is detected by the WMD 104, the WMD 104 may issue the audible alert in the first language via the medical device speaker 118.

The audible alert refers to a sound, a voice, or a spoken notification intended to convey important information to the user 116. Further, the first language refers to a default language used by the WMD 104 to communicate audible alerts, to the user 116. The first language is a language in which the WMD 104 may be set to deliver the audible alerts. For example, if the WMD 104 is configured to deliver the audible alerts in English, which may be the first language, then all the audible alerts, like warnings about an impending shock, may be issued in English. Further, if the WMD 104 is set to Spanish as the first language, the audible alerts may be issued in Spanish by default.

In some embodiments, the physician may be assigned to the patient 116 to provide medical advice and manage ongoing health monitoring of physiological parameters through the WMD 104. In an example, the physician may be any of but is not limited to a cardiologist, a general practitioner (GP), an emergency medicine physician, an electro physicist, or a general physician assistant. The physician may interpret patient data collected by the WMD 104, such as but not limited to heart rate, blood pressure, and parameters corresponding to any detected cardiac conditions.

In some embodiments, the WMD 104 may be an ASSURE WMD provided by Kestra Medical Technologies Inc., Kirkland WA (Kestra), but in other embodiments the WMD 104 may be provided by other companies. So, while the following description references Kestra as the WMD provider, other embodiments are not limited to using Kestra as the WMD provider. In some embodiments, the WMD 104 and mobile device 106 are similar to those described in U.S. Pat. Nos. 8,838,235, 11,235,143, 11,794,005, and 11,950,174, all of which are incorporated by reference herein for all purposes.

The WMD 104 may be communicatively coupled to the mobile device 106 via the network 108 for transmitting and receiving various data. By way of an example, the WMD 104 may transmit a pre-alert signal, an information indicative of an audio output state of the WMD 104, an acknowledgement message, and/or the audible alert in the first language to the mobile device 106 via the network 108. By way of another example, the WMD 104 may receive a suppression communication/suppression signal from the mobile device 106 via the network 108. In some implementations, a connection between the WMD 104 and the mobile device 106 may be established via a Bluetooth Low Energy (BLE) wireless connection.

The user 116 may interact with the mobile device 106 to review real-time patient data based on the physiological parameters. The user 116 may further receive alerts or notifications, and interact with medical professionals using the mobile device 106. The user 116 may be required to provide feedback or consent corresponding to the medical system 100 through the mobile device 106.

Further, the mobile device 106 may be used by a user 116 to log into and interact with computing platforms or interfaces provided by the server 102. A computing platform may execute applications according to implementations of the present disclosure. Examples of the mobile device 106 may include a notebook, a desktop, a netbook, smartphones, laptops, a tablet, voice-enabled devices, a smart device, and/or the like. It is contemplated that implementations of the present disclosure may be realized with any appropriate type of mobile device. In some examples, the mobile device 106 may include a mobile application executed thereon, which may be used to display one or more pages of the computing platform executing applications. In some examples, the mobile device 106 may display one or more User Interfaces (UIs) or Graphical UIs that enable the user 116 to interact with the computing platforms. A memory (not shown) of the mobile device 106 may store the application installed in the mobile device 106. When the application is run or executed, necessary files are loaded into the memory for quick access. The memory allows a processor (not shown) of the mobile device 106 to retrieve and manipulate data efficiently while the application is running or in use.

In an example, the application may be Field Service Lightning (FSL) available from Salesforce. FSL may be customized to suit the intended purpose of the application. In another example, the application may be the ASSURE Patient App available from Kestra. The user 116 may download the ASSURE Patient App (also referred to herein as mobile application) in the mobile device 106 and launch the mobile application.

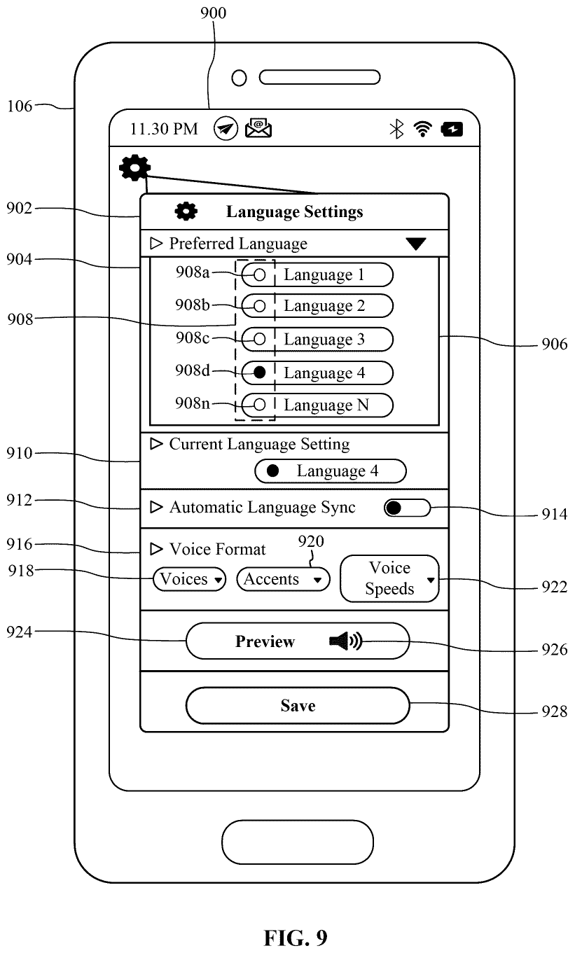

The mobile device 106 may include an Input/Output (I/O) unit 120. By way of an example, the user 116 may use the I/O unit 120 of the mobile device 106 to provide an input to the mobile application running on the mobile device 106 and/or receive an output accordingly. The input may be then sent to the server 102 for processing at the back-end. The I/O unit 120 may include a keypad or a touchpad 122, and a microphone 124 to provide input to the mobile application running on the mobile device 106. For example, the user 116 may select the second language through a language setting option on an interface of the mobile application running on the mobile device 106 by providing the input through the keypad or the touchpad 122. The input may include, but is not limited to, a selection of a language, the pre-alert signal, the information related to audio output states of the WMD 104, the audible alert in the first language, user feedback, and/or the like. The I/O unit 120 may further include at least one mobile device speaker 126 to output the audible alert in the second language. The output may include, but is not limited to, an audible alert in the second language. The second language refers to any language that is used to mirror or translate the audible alert in the first language issued by the WMD 104 for the user 116 who does not prefer or speak the first language or the default language. The second language may be selected by the user 116 in the application installed on the mobile device 106, or selected automatically based on language of an operating system of the mobile device 106. For example, when the second language is Spanish, the mobile device speaker 126 may output the audible alert in Spanish. The selection of the second language is further described in detail in FIG. 9.

In some embodiments, the I/O unit 120 may further include a display to display a visual alert in the second language corresponding to the audible alert. Various examples of outputting the alerts in the second language are described in conjunction with FIGS. 2-9.

FIG. 2 illustrates an example architecture of the language mirroring engine 114 coupled to a database, within the server 102 disclosed in FIG. 1, according to an embodiment of the present disclosure. FIG. 2 is described in conjunction with FIG. 1.

The language mirroring engine 114 may perform various operations for outputting the audible alert in the second language. To perform the operations, the language mirroring engine 114 may include a prompt recognition module 202 (or a voice recognition module 202), a notification module 204, a data extraction module 206, and an Input/Output (I/O) module 208. The language mirroring engine 114 may be communicatively coupled to a database 210 that stores various data and intermediate results generated by the modules 202-208.

In an implementation, the prompt recognition module 202 may receive an indication of the audible alert output by the medical device speaker 118 of the WMD 104 in the first language. The indication may be a voice prompt, a pre-alert signal, and/or an audio output status stream. In particular, the indication in the first language may be received by the microphone 124 and further processed by the processor of the mobile device 106 and interpreted by the server 102 at back-end. The server 102 may receive the indication via the prompt recognition module 202 of the language mirroring engine 114 or the application running on the mobile device 106. Further, in some implementations, the prompt recognition module 202 may recognize the audible alert in the first language. The recognized audible alert in the first language may be further transmitted to other modules for translation.

If the indication includes the voice prompt or the audible alert in the first language or the audio output status stream, the data extraction module 206 may extract a corresponding audible alert in the second language from a Look Up Table (LUT) 212 within the database 210. The LUT 212 may be utilized for mirroring or translation of the audible alert from the first language to the second language. The LUT 212 may store precomputed translations or mirrored versions of audible alerts in various languages. By referencing the LUT 212, the language mirroring engine 114 may access the appropriate translated audible alerts without requiring real-time translation processing, significantly improving efficiency and speed of communication in urgent medical situations.

The LUT 212 may include a variety of entries, each corresponding to a specific audible alert generated by the WMD 104. The audible alerts may include important warnings or alerts including device-related conditions or conditions associated with patient health, such as low battery warnings, arrhythmia detection, or imminent shocks. For each audible alert in the first language, the LUT 212 stores one or more translations in different languages to ensure that a message is clearly communicated to patients from diverse linguistic backgrounds.

Furthermore, the LUT 212 may be updated dynamically, which enhances the scalability and flexibility of the medical system 100. New audible alerts may be added to the LUT 212 as the medical system 100 evolves, and additional languages may be incorporated as needed. The dynamic updating capability ensures that the medical system 100 remains relevant to a growing, diverse patient base and may adapt to new medical conditions or updates to functionality of the WMD 104.

By using the LUT 212, the medical system 100 avoids a need for real-time language translation, which may be computationally expensive and time-consuming. Instead, the LUT 212 enables quick retrieval of pre-computed translations, resulting in faster system response times and enhanced user experience. Efficiency of the LUT 212 also ensures that the medical system 100 may operate in high-pressure medical situations, where time-sensitive alerts and instructions are essential for patient safety. An example of the LUT 212 is provided as per table (1) given below:

TABLE 1
Look Up Table (LUT)

| Voice | Audible Alerts | | |
|---|---|---|---|
| prompt ID | Language 1 | Language 2 | Language N |
| Low Battery | Please replace the | Veuillez remplacer | Por favor reemplace la |
| | battery. | la batterie. | batería. |
| Arrhythmia | An arrhythmia has | Une arythmie a été | Se detectó una arritmia. |
| Detection | been detected. | détectée. | |
| Impending | Shock is imminent. | Un choc est | El choque es inminente. Por |
| Shock | Please press the alert | imminent. Veuillez | favor presione el botón de |
| | button. | appuyer sur le | alerta. |
| | | bouton d'alerte. | |
| Device | Device malfunction | Défaillance de | Se detectó una falla en el |
| Malfunction | detected. Please | l'appareil détectée. | dispositivo. Por favor revise |
| | check the device. | Veuillez vérifier | el dispositivo. |
| | | l'appareil. | |

The example of LUT 212, as shown in the table (1), depicts that the LUT 212 stores different audible alerts in multiple languages (e.g., language 1, language 2, and language N). Each row in the table (1) represents a different condition or alert, and each column includes a corresponding audible alert message in different languages. For example, different voice prompts are listed below:

Low Battery: An audible alert for low battery in language 1 (e.g., English) is “Please replace the battery.” The same audible alert has corresponding translated audible alerts in language 2 (e.g., French) as “Veuillez remplacer la batterie,” and in language N (e.g., Spanish) as “Por favor reemplace la batería.”

Arrhythmia Detection: An audible alert for arrhythmia detection in language 1 (e.g., English) is “An arrhythmia has been detected,” which has corresponding translations in language 2 (e.g., French) as “Une arythmie a été détectée” and in language N (e.g., Spanish) as “Se detectó una arritmia.”

Impending Shock: An audible alert for impeding shock in language 1 (e.g., English) is “Shock is imminent. Please press the alert button,” which has corresponding translations in language 2 (e.g., French) as “Un choc est imminent. Veuillez appuyer sur le bouton d'alerte,” and in language N (e.g., Spanish) as “El choque es inminente. Por favor presione el botón de alerta.”

Device Malfunction: An audible alert for impending shock in language 1 “Device malfunction detected. Please check the device,” which has corresponding translations in language 2 (e.g., French) as “Défaillance de l'appareil détectée. Veuillez vérifier l'appareil,” and in language N (e.g., Spanish) as “Se detectó una falla en el dispositivo. Por favor revise el dispositivo.”

For example, the WMD 104 may detect low battery, and triggers an audible alert in language 1 (e.g., English): “Please replace the battery.” If the user 116 has selected language 2 (e.g., French) on the application running on the mobile device 106 (or the language mirroring engine 114), the data extraction module 206 may use the LUT 212 of table (1) to retrieve the French translation: “Veuillez remplacer la batterie.” If the user 116 selects language 3 (e.g., Spanish) on the application running on the mobile device 106 (or the language mirroring engine 114), the data extraction module 206 may use the LUT 212 of table (1) to retrieve the corresponding translation “Por favor reemplace la batería” from the LUT 212.

In some implementations, the language mirroring engine 114 may be configured to provide simple or alternative wording for better clarity, the LUT 212 may also include alternate versions of the audible alerts. For example, instead of “Please replace the battery,” the medical system 100 may issue an alternative audible alert “Replace the battery” in English. Therefore, the language mirroring engine 114 on the server 102 may push or allow different ways of delivering audible alerts on the application running on the mobile device 106.

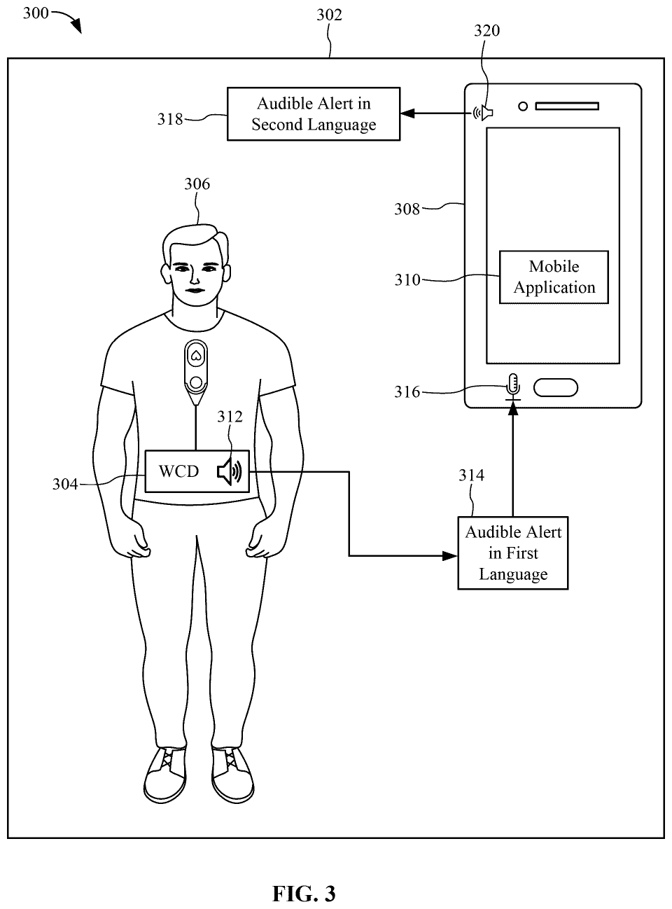

Further, if the indication is a pre-alert signal from the WMD 104, the notification module 204 may transmit a suppression communication or suppression signal to the WMD 104 for suppressing the audible alert in the first language. In such a case, the prompt recognition module 202 may receive an acknowledgement message from the WMD 104, in response to the transmission of the suppression communication or the suppression signal. After receiving the acknowledgement message, the data extraction module 206 may perform its operation in a similar way as explained above. In some cases, when the acknowledgement message is not received, the data extraction module 206 may perform its operation after a second preset time (e.g., 1s, 2s, 5s, and/or the like). Different scenarios of generating the audible alerts when the indication is audible alert, pre-alert signal, and/or the audio output status stream are explained in detail in conjunction with FIGS. 3-6.

Further, the I/O module 208 may render the audible alert in the second language extracted by the data extraction module 206 to the user 116 via the mobile device speaker 126 of the mobile device 106.

FIG. 3 illustrates an example scenario 300 of outputting an audible alert in a second language upon recognition of the audible alert in a first language, according to an embodiment of the present disclosure. FIG. 3 is described in conjunction with FIGS. 1 and 2. The example scenario 300 depicts a medical system 302 which is analogous to the medical system 100. As illustrated in FIG. 3, the medical system 302 includes a Wearable Cardiac Device (WCD) 304 worn by a patient 306 and a smartphone 308. It may be noted that in the scenario 300 or the medical system 302, the WCD 304 may correspond to one of WMDs (e.g., WMD 104 depicted in FIG. 1), the patient 306 may correspond to one of users (e.g., user 116 depicted in FIG. 1), and the smartphone 308 may correspond to one of mobile devices (e.g., mobile device 106 depicted in FIG. 1).

The WCD 304 may be communicatively coupled with the smartphone 308, and a mobile application 310 may be installed and/or running on the smartphone 308. It may be noted that the mobile application 310 may be hosted on the server 102 and includes the language mirroring engine 116 to perform various operations of outputting the audible alert in the second language (depicted in FIG. 1). The medical system 302 ensures that audible alerts (e.g., voice prompts) issued by the WCD 304, which are in the first language or a pre-defined or default language, are translated and mirrored into the second language that the patient 306 understands and prefers.

Further, the WCD 304 may monitor cardiac condition of the patient 306. In particular, the WCD 304 may detect various conditions associated with the patient 306 such as arrhythmia (e.g., atrial fibrillation, or ventricular tachycardia), bradycardia, and/or other abnormal heart rhythms. Also, the WCD 304 may detect other physiological events such as oxygen desaturation, elevated heart rate, and/or increased blood pressure. Additionally, the WCD 304 may be capable of detecting device-related conditions, such as low battery states, device malfunction, sensor disconnection, and/or poor contact between monitoring/therapy electrodes (not shown) of the WCD 304 and skin of the patient 306. The conditions associated with the patient 306 and the device-related conditions are critical in alerting the patient 306 for potential issues with either the health of the patient 306 or functionality of the WCD 304, prompting necessary actions to ensure safety of the patient 306 and/or effectiveness of the WCD 304.

After detecting the above-mentioned conditions associated with the patient 306 and/or the device, the WCD 304 may generate corresponding audible alerts. The audible alerts are intended to notify the patient 306 about the detected conditions and instruct the patient 306 to perform specific actions, such as replacing a battery or pressing an alert button in response to the detection of arrhythmia. The audible alerts issued by the WCD 304 are in the first language, or the pre-defined or default language. The audible alerts in the first language are broadcast through a medical device speaker 312 (analogous to the medical device speaker 118) integrated into the WCD 304. By way of an example, the medical device speaker 312 may issue an audible alert in a first language 314.

The WCD 304 may be wirelessly coupled to the smartphone 308 and the smartphone 308 may act as a platform for running the mobile application 310 hosted on the server 102 depicted in FIG. 1. The smartphone 308 includes a microphone 316 (similar to the microphone 124) that may receive the audible alerts in the first language issued by the WCD 304 via the medical device speaker 312. For example, the microphone 316 may receive the audible alert in the first language 314 issued by the WCD 304 via the medical device speaker 312.

It may be noted that the communication between the server 102 and the smartphone 308 allows the smartphone 308 to interface with the server 102 for sending, receiving, and displaying relevant patient information, including the audible alerts, and other necessary instructions. For example, from the microphone 316, the received audible alert in the first language 314 may be sent to the mobile application 310. The mobile application 310 may recognize the audible alert in the first language 314.

Further, the mobile application 310, upon recognition of the audible alert in the first language 314, may translate the audible alert in the first language 314 into the second language that the patient 306 understands or prefers. As a result of the translation, the audible alert in the second language 318 may be generated. The mobile application 310 ensures that the translation is executed in such a way that the patient 306 is able to take necessary action upon receiving the audible alert in the second language 318. In particular, the prompt recognition module 202 of the language mirroring engine 116 within the mobile application 310 may recognize the audible alert in the first language 314. Once the audible alert in the first language 314 is recognized, the mobile application 310 retrieves a corresponding translation from the LUT 212 using the data extraction module 206 depicted in FIG. 2. The LUT 212 stores mapping of the audible alerts in the first language with the second language and potentially with other languages (e.g., Languages 3, 4, etc.). The LUT 212 may be indexed by the recognized audible alerts, allowing the mobile application 310 to select a correct translated audible alert to be output in the second language, ensuring that the audible alerts are accurately mirrored in the second language that the patient 306 understands, enhancing communication and improving patient compliance.

For example, the mobile application 310 may retrieve the audible alert in the second language 318 from the LUT 212. The second language may be selected by the patient 306 or automatically selected by the mobile application 310. In some embodiments, the patient 306 may select the second language manually through a language setting of the mobile application 310 by providing input through the keypad/touchpad 122 on the smartphone 308. Alternatively, in some other embodiments, the mobile application 310 may automatically detect a language of an operating system of the smartphone 308 and select that automatically detected language as the second language. The selection of the second language is further explained in detail in conjunction with FIG. 9. The automatic detection may ensure that the medical system 302 is easy to use without requiring any manual input from the patient 306.

Further, the medical system 302 may also have a feature of mirroring the audible alerts in the same language but with different voice formats. The feature may be useful in scenarios where the patient 306 may benefit from repetition or reinforcement of a message. For example, if the patient 306 is instructed to press a button during a shock alarm, repeating the audible alert in the same language but with different words or emphasis may enhance patient's understanding and compliance with the action.

Once the audible alert in the second language 318 or in different voice format is generated, a mobile device speaker 320 (analogous to the mobile device speaker 126) of the smartphone 308 may output the audible alert in the second language 318 or in different voice format. The audible alert in the second language 318 ensures that the patient 306 consumes the audible alert effectively, enabling the patient 306 to receive clear and comprehensible notifications regarding health conditions or device-related conditions. In some implementations, the audible alert in the second language 318 may be consumed by a bystander or a caregiver, to receive clear and comprehensible notifications regarding health conditions of the patient 306 or device-related conditions notifications. The audible alert may include information such as detected arrhythmias, device malfunctions, and/or other health indicators. Alternatively, the audible alert may be presented in various voice formats, such as different tones, pitches, and/or speech styles, to enhance the clarity of the audible alert. The voice formats may be tailored to preferences or needs of the patient 306, bystander, and/or caregiver, thereby improving the ability of the patient 306, bystander, and/or caregiver to recognize and respond to the audible alert. In some implementations, the mobile application 310 may be capable of detecting or learning a tone, pitch, or speech style of the patient 306, bystander, and/or caregiver to identify an appropriate tone, pitch, or speech style for the audible alert.

The patient 306 may then perform an action based on information conveyed through the audible alert in the second language 318 or the different voice formats. The information may be about adjusting the device, taking medication, and/or seeking immediate medical attention. The multi-modal communication ensures that the patient 306 is fully engaged and may act swiftly and appropriately in response to the audible alert in the second language 318 or different voice formats.

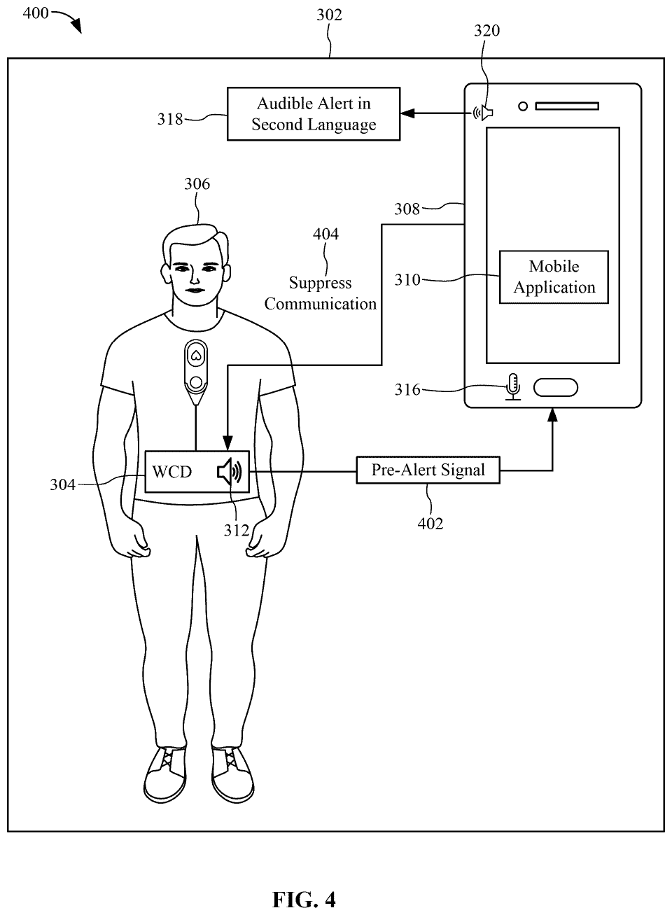

FIG. 4 illustrates an example scenario 400 of suppressing the audible alert in the first language 314 and outputting the audible alert in the second language 318, in accordance with an embodiment of the present disclosure. FIG. 4 is described in conjunction with FIGS. 1-3.

In the scenario 400, the audible alert in the first language 314 generated by the WCD 304 (as described in FIG. 3) may be suppressed, and only the audible alert in the second language 318 may be provided to the patient 306 through the mobile application 310 installed on the smartphone 308. The suppression may improve patient compliance and communication by ensuring that the audible alert is delivered in a language that the patient 306 may easily understand. For each audible alert that the WCD 304 is configured to issue, a corresponding pre-alert signal 402 may be generated and transmitted by the WCD 304 before transmitting the audible alert in the first language 314, to the smartphone 308.

In some implementations, the pre-alert signal 402 may be inaudible to humans. Further, the pre-alert signal 402 may have a frequency and/or amplitude that may not be heard by the patient 306 but may be detected by the smartphone 308. In an example, frequencies above 20 kHz are not audible to humans but can be detected by the smartphone 308. For instance, the smartphone 308 can pick up ultrasonic signals for sound-based communication between devices. In another example, frequencies below 20 Hz, known as infrasound, are also inaudible to humans, but the microphone 316 of the smartphone 308 can detect them. Further, regarding amplitude, audible alerts with very low amplitudes might not be audible to humans but could still be detected by the microphone 316 of the smartphone 308. Additionally, the pre-alert signal 402 serves as a precursor or notification to the mobile application 310 that an impending audible alert (e.g., the audible alert in the first language 314) is about to be issued by the WCD 304.

Further, the mobile application 310 may receive the pre-alert signal 402. Upon receiving the pre-alert signal 402, the mobile application 310 may send a suppression communication 404 including instructions to the WCD 304. In some examples, the suppression communication 404 may refer to a radio frequency being utilized to communicate with the WCD 304 (e.g., via Bluetooth, BLE, WiFi, and the like). It may be noted that the suppression communication 404 may correspond to a suppression signal, therefore the terms “suppression communication” and “suppression signal” are used interchangeably in the present disclosure. The suppression instructions may direct the WCD 304 to refrain or not to issue the audible alert in the first language 314. Instead, the mobile application 310 may take full control of issuing audible alerts, providing the audible alert in the second language 318, which is patient's preferred language. The mobile application 310 may either provide the translated audible alert in the second language 318 via the mobile device speaker 320 immediately or after a preset time delay (e.g., 1 s, 2 s, 5 s, 10 s, and/or the like) or depending on the configurations of the medical system 302.

In an alternative embodiment, the WCD 304 may send an acknowledgment message back to the smartphone 308 after receiving the suppression communication 404. The acknowledgment message informs the mobile application 310 that the WCD 304 may not issue an original audible alert (e.g., the audible alert in the first language 314). After receiving the acknowledgment message, the mobile application 310 may immediately issue the audible alert in the second language 318 via the mobile device speaker 320 of the smartphone 308, ensuring that the patient 306 receives the audible alert in the second language 318 preferred by the patient 306 without any delay. The suppression of the audible alert in the first language 314 enhances user experience by ensuring that the patient 306 only hears the translated audible alert in the second language 318, eliminating potential confusion and reinforcing effectiveness of the audible alert. Additionally, the use of the pre-alert signal 402 and an ability to suppress the audible alert in the first language 314 ensures seamless integration between the WCD 304 and the mobile application 310, improving overall patient engagement and compliance with the medical system 302.

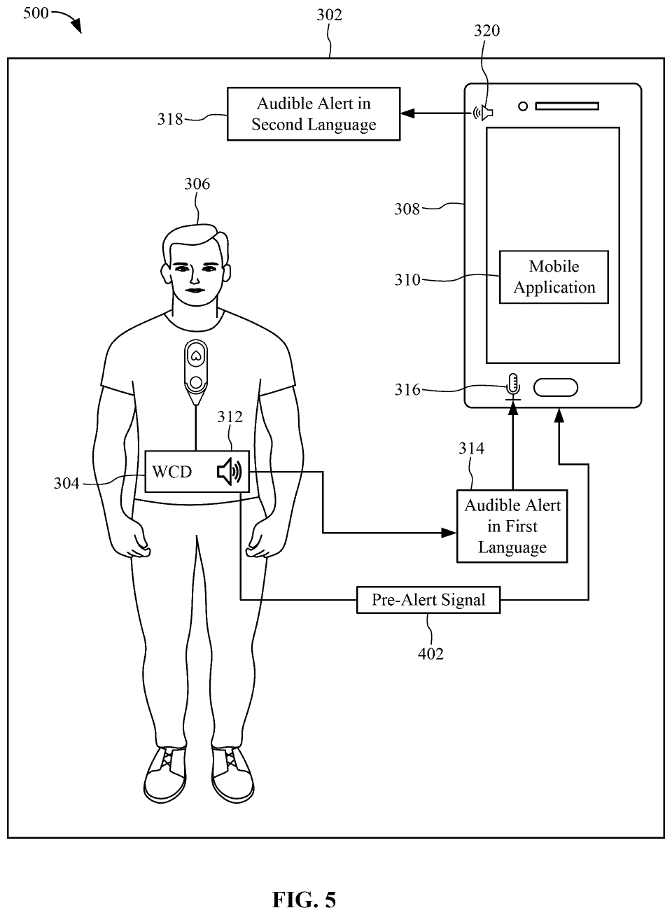

FIG. 5 illustrates an example scenario 500 of outputting the audible alert in the second language 318 in response to receiving the pre-alert signal 402 from a WMD, according to an embodiment of the present disclosure. FIG. 5 is described in conjunction with FIGS. 1-4.

Referring back to FIG. 4, when the mobile application 310 sends the suppression communication 404 within the preset time after receiving the pre-alert signal 402 from the WCD 304, the WCD 304 refrains from issuing the audible alert in the first language 314. Instead, the mobile application 310 takes over and issues the audible alert in the second language 318. However, as illustrated in the scenario 500, when the WCD 304 fails to receive the suppression communication 404 from the mobile application 310 within a first preset time, the WCD 304 may proceed to issue the audible alert in the first language 314. In addition to issuing the audible alert in the first language 314, the mobile application 310 may also issue the audible alert in the second language 318 after a second preset time from receiving the pre-alert signal 402. A value of the second preset time may be longer than the first preset time. For example, the first preset time may be 5 seconds, while the second preset time may be 10 seconds. In such example, if the suppression communication 404 is not received within 5 seconds of transmitting the pre-alert signal 402, the WCD 304 may issue the audible alert in the first language 314 immediately after the 5-second time duration ends. In other words, the WCD 304 may wait for 5 seconds to receive the suppression communication 404 after transmitting the pre-alert signal 402. If the suppression communication is not received from the mobile application 310 of the smartphone 308 within 5 seconds, the WCD 304 may proceed to output the audible alert in the first language 314. Further, after the completion of 10 seconds, the mobile application 310 may issue the audible alert in the second language 318.

In some embodiments, the mobile application 310 may automatically issue the audible alert in the second language 318 after expiration of the second preset time from receiving the pre-alert signal 402, regardless of whether the WCD 304 issues the audible alert in the first language 314. In such embodiments, once the mobile application 310 detects the pre-alert signal 402, the mobile application 310 may issue the audible alert in the second language 318 after the second preset time. During the second preset time, the mobile application 310 may not respond to or recognize the audible alert in the first language 314 from the WCD 304, as the mobile application 310 may be actively mirroring the audible alert in the second language 318, ensuring that only the translated audible alert in the second language 318 is heard by the patient 306, avoiding any confusion caused by multiple audible alerts in different languages. In an alternative embodiment, the mobile application 310 may not mirror the audible alert. Instead, the mobile application 310 may issue a single audible alert.

If the mobile application 310 receives the pre-alert signal 402 and sends the suppress communication or instruction 404, the audible alert in the second language 318 may be issued by the mobile application 310. Further, if the mobile application 310 does not detect the pre-alert signal 402 or fails to send the suppress communication 404, the WCD 304 may issue the audible alert in the first language 314. In such embodiment, the medical system 302 only produces one audible alert, either in the first language or the second language, depending on the action taken by the mobile application 310, providing flexibility in how the audible alerts are issued, allowing the medical system 302 to adapt to different user preferences and scenarios.

The patient 306 may always receive the audible alert in the preferred language, improving communication, comprehension, and compliance with health-related instructions. The medical system 302 ensures that the WCD 304 and the mobile application 310 operate together seamlessly to deliver the most effective alert system based on the needs of the patient 306, whether by mirroring the audible alert in the second language 318 or by issuing the audible alert in a timely manner based on configurations.

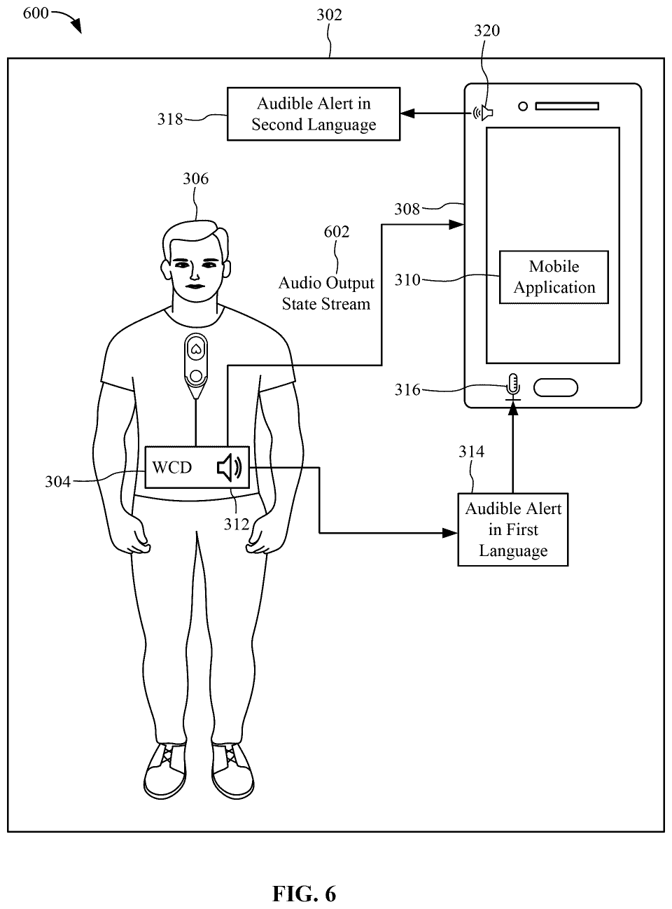

FIG. 6 illustrates an example scenario 600 of outputting the audible alert in the second language 318 upon detecting an audio output state of a WMD, according to an embodiment of the present disclosure. FIG. 6 is described in conjunction with FIGS. 1-5.

In the scenario 600, instead of the mobile application 310 listening to audio inputs to recognize voice prompts, the mobile application 310 may recognize a transition of the WCD 304 into an audio output state associated with a particular voice prompt (e.g., the issuance of the audible alert in the first language 314). The transition occurs when the WCD 304 starts streaming an audio file (e.g., a Waveform audio file (WAV file)) corresponding to the audible alert in the first language 314. Examples of the audio file may include, but are not limited to, an MPEG-1 Audio Layer 3 (MP3) file, the WAV file, an Advanced Audio Codec (AAC) file, and/or a Free Lossless Audio Codec (FLAC) file. The mobile application 310 may accurately recognize when the audible alert in the first language 314 is being issued without requiring to analyze an audio signal, simplifying the process, and reducing the chance for errors. The WCD 304 may be configured to transmit information indicative of the audio output state (e.g., an audio output state stream 602) to the mobile application 310 when the smartphone 308 is paired and connected to the WCD 304. In some embodiments, the connection between the WCD 304 and the smartphone 308 is established via a Bluetooth Low Energy (BLE) wireless connection, Bluetooth classic, or any other electromagnetic communication protocol.

Upon receiving the audio output state stream 602, the mobile application 310 may recognize the audio output state corresponding to the audible alert in the first language 314. Once the mobile application 310 recognizes the audio output state, the mobile application 310 may issue the corresponding audible alert in the second language 318 after the WCD 304 outputs the audible alert in the first language 314.

In some embodiments, the WCD 304 described in the medical system 302 may be replaced with other types of WMDs that serve similar or complementary purposes. For example, a Wearable Electrocardiogram (ECG) device may be one of the WMDs (e.g., WMD 104) that may be used in place of the WCD 304. The wearable ECG device continuously monitors electrical activity of heart of the patient 306, providing valuable real-time data for detecting various cardiac events, such as arrhythmias or heart rate abnormalities. Similar to the WCD 304, the wearable ECG device may be configured to issue the audible alerts to the patient 306 when certain conditions or thresholds are detected, such as an abnormal heart rhythm or other critical health events.

The audible alerts in the alternative WMDs may function similarly to those of the WCD 304, alerting the patient 306 to take necessary actions such as adjusting the WMD, seeking medical assistance, or following prescribed treatment steps. Examples of the alternative WMDs include, but are not limited to, wearable glucose monitor, blood pressure monitor, or any device capable of detecting physiological conditions, may also be incorporated into the medical system 302. The alternative medical device may communicate with the smartphone 308 and the mobile application 310 to issue the audible alerts to the patient 306 based on the data detected, providing real-time feedback and instructions in the preferred language of the patient 306.

In some embodiments, the WCD 304 may be configured to stream data indicative of the audible alert in the first language 314 that the WCD 304 is outputting or is about to output to the mobile application 310. The streaming of the data may be accomplished by transmitting a pre-alert signal (e.g., the pre-alert signal 402) or a message to the mobile application 310 that indicates an imminent issuance of the audible alert in the first language 314, triggered by a specific event. For example, when the WCD 304 detects a particular condition (e.g., arrhythmia, low battery, or sensor disconnection), the WCD 304 may send the pre-alert signal 402 to the mobile application 310, notifying the mobile application 310 that a specific audible alert or voice prompt is about to be generated. Upon receiving the pre-alert signal 402, the mobile application 310 may be able to preemptively prepare for the corresponding audible alert or the voice prompt. Specifically, the mobile application 310 may initiate translation of the audible alert in the first language 314 into the second language (preferred language of the patient 306) prior to actual output of the audible alert in the first language 314 or the voice prompt by the WCD 304. The streaming of the data enables the mobile application 310 to output the translated audible alert in the second language 318 without delay, thereby improving the overall responsiveness and effectiveness of the medical system 302.

In some embodiments, the mobile application 310 may be configured to request language settings of the WCD 304 directly, or alternatively, the language settings of the WCD 304 may be included within the audio output state stream 602 sent to the mobile application 310, along with an audio file identifier (a unique reference or identifier of the audio file) corresponding to the audible alert in the first language 314. Examples of the audio file may include, but are not limited to, an MPEG-1 Audio Layer 3 (MP3) file, a Waveform Audio file (WAV file), an Advanced Audio Codec (AAC) file, a Free Lossless Audio Codec (FLAC) file, and/or the like. This enables the mobile application 310 to verify whether the audible alert or the voice prompt mirrored and output by the mobile application 310 is in the second language, as preferred by the patient 306, rather than in the first language. This functionality is particularly useful in embodiments where the second language is automatically selected based on the operating system's language of the smartphone 308, ensuring that the patient 306 consistently receives the correct translation in the preferred language, regardless of the first or initial language of the WCD's output.

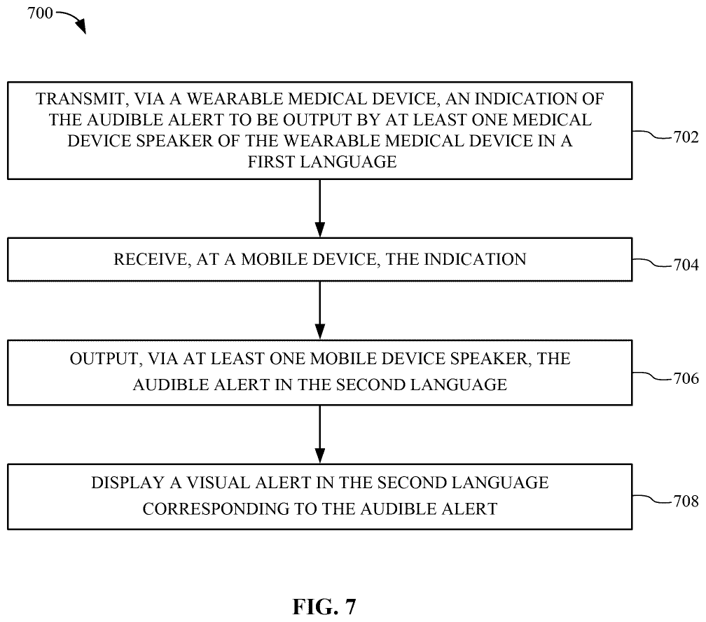

FIG. 7 illustrates an example method 700 for outputting an audible alert in a second language, according to an embodiment of the present disclosure. The method 700 is executed by the medical system 100. FIG. 7 is explained in conjunction with FIGS. 1-6. Although an example utilized for method 700 depicts a particular sequence of operations, the sequence may be altered without departing from the scope of the present disclosure. For example, some of the operations depicted in FIG. 7 may be performed in parallel or in a different sequence that does not materially affect a function of the method 700. In other examples, different components of the medical system 100 that executes the method 700 may perform the operations substantially in parallel or in a specific sequence.

At step 702, the method 700 includes transmitting, via the WMD 104, an indication of the audible alert to be output by the at least one medical device speaker 118 of the WMD 104 in a first language. The WMD 104 may be configured to provide therapy to a patient (e.g., patient 306) upon detection of a cardiac condition of the patient 306, such as detecting a cardiac condition like arrhythmia, tachycardia, or bradycardia based on patient data. The first language may be a standard language in which the WMD 104 always outputs the audible alert. The first language may be a predefined standard language in which the WMD 104 consistently outputs audible alerts. For example, the first language may be English as an example, although other languages may be supported based on specific configuration of the WMD 104.

At step 704, the method 700 includes receiving, at the mobile device 106, the indication from the WMD 104. The indication may be the audible alert output by the at least one medical device speaker 118. In an example, the indication may be the audible alert itself, as output by the medical device speaker 118 of the WMD 104. In another example, the indication may be a pre-alert signal, which is an early notification of the audible alert in the first language. The pre-alert signal may be output by the medical device speaker 118. The pre-alert signal may be transmitted via sound waves which have a frequency and/or amplitude inaudible to humans. Alternatively, the pre-alert signal may be transmitted as an electronic communication directly from the WMD 104 to the mobile device 106. In the context of the present disclosure, the electronic communication may refer to electromagnetic communication involving the transmission and reception of data via electromagnetic waves (such as radio frequency waves). The pre-alert signal serves as an early trigger for the mobile device 106 to prepare for a forthcoming alert.

At step 706, the method 700 includes outputting, via at least one mobile device speaker 126, the audible alert in the second language. The second language may be selected by the patient 306, or determined automatically based on a language setting of the mobile device 106 (as further detailed in FIG. 9). The second language may be used to mirror the audible alert generated by the WMD 104 in the first language, ensuring that the patient 306 receives clear and understandable notifications in the second language or a language that the patient 306 is familiar with. Examples of the second language may include, but are not limited to, Spanish, Chinese, Arabic, Hindi, Japanese, French, and/or a user-preferred language or a language based on a geographical location. The audible alert may include alerts associated with WMD conditions and/or alerts associated with patient data. For example, the audible alert output by the mobile device speaker 126 may include various types of notifications, such as alerts related to WMD conditions (e.g., low battery, device malfunction, and/or the like) or patient-related data (e.g., detected arrhythmia, abnormal heart rate, and/or the like).

At step 708, the method 700 includes displaying a visual alert in the second language corresponding to the audible alert in the second language. The visual alert may provide supplementary information to the patient, ensuring that an alert is communicated effectively through multiple channels (e.g., auditory and visual). In other words, when an alert is required to be output due to a health condition, the method 700 may include rendering the audible alert and/or the visual alert in the second language to ensure clear communication. For example, a screen of a UI of the mobile device 106 may display a message in the second language alongside the audible alert.

By way of an example, consider a scenario where the WMD 104 detects a dangerously low blood oxygen level (below 90%). In such a scenario, the mobile device 106, upon receiving the indication from the WMD 104, may display the visual alert in the second language, such as, “Advertencia: El nivel de oxígeno está por debajo del 90%. Por favor, busque atención médica inmediata,” corresponding to the audible alert in the second language. In some examples, the visual alert and the audible alert in the second language are simultaneously output by the mobile device 106. Displaying the visual alert facilitates patients who may be more comfortable with one language (e.g., the second language) over the other language (e.g., the first language), ensuring the patients receive important health information in an understandable language (e.g., the second language) quickly and effectively. Here, for brevity, one example scenario is explained, however the method 700 may be applicable for other conditions associated with the patient data such as high heart rate, low blood sugar, and/or other conditions associated with the patient data or the WMD-related conditions. Steps 702-708 are already explained in detail in conjunction with FIGS. 1-6, therefore, for brevity, repeated description is omitted from FIG. 7.

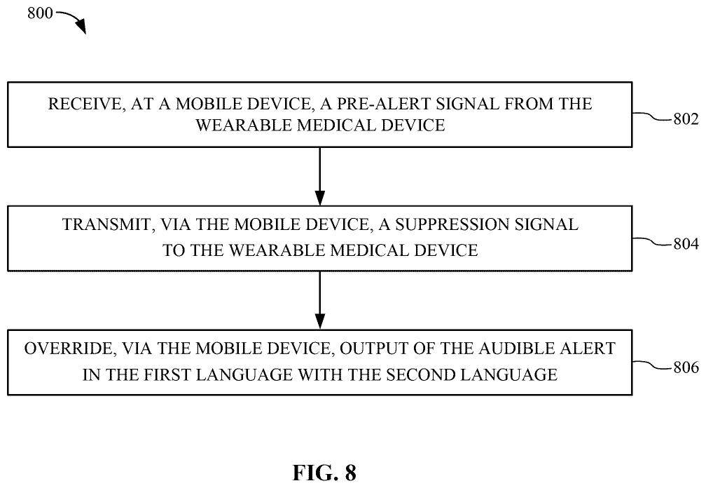

FIG. 8 illustrates an example method 800 for overriding an audible alert in a first language with the audible alert in a second language, according to an embodiment of the present disclosure. The method 800 is executed by the medical system 100. FIG. 8 is explained in conjunction with FIGS. 1-7. Although an example utilized for method 800 depicts a particular sequence of operations, the sequence may be altered without departing from the scope of the present disclosure. For example, some of the operations depicted in FIG. 8 may be performed in parallel or in a different sequence that does not materially affect a function of the method 800. In other examples, different components of the medical system 100 that executes the method 800 may perform the operations substantially in parallel or in a specific sequence.

At step 802, the method 800 includes receiving a pre-alert signal 402, at the mobile device 106, from the WMD 104. In embodiments, the pre-alert signal 402 serves as an early indication that a critical alert, such as an impending shock or system malfunction, is about to be issued by the WMD 104 in the first language (e.g., a default language of the WMD 104). The pre-alert signal 402 may be inaudible to the patient 306 but is detectable by the mobile device 106, which is wirelessly connected to the WMD 104. The pre-alert signal 402 ensures that the mobile device 106 is prepared to take action before the WMD 104 produces the audible alert in the first language.

Further, at step 804, the method 800 includes transmitting, via the mobile device 106, a suppression signal (e.g., 404) to the WMD 104. The suppression signal 404 instructs the WMD 104 to delay or suppress the issuance of the audible alert in the first language. By suppressing or postponing the audible alert in the first language, the mobile device 106 may ensure that the audible alert is then presented in the second language (preferred language of the patient 306), which enhances patient comprehension and response time.

At step 806, the method 806 includes overriding, via the mobile device 106, output of the audible alert in the first language with the second language. Step 806 is carried out through a mobile application (e.g., 310) hosted on the server 102 which is designed to listen for incoming signals from the WMD 104. Once the suppression signal 404 is received and acknowledged by the WMD 104, the mobile application 310 triggers the corresponding audible alert in the second language. The mobile application 310 accomplishes overriding by referencing a lookup table (e.g., 212) stored in the database 210. The lookup table 212 includes mappings between specific alert triggers (e.g., low battery, arrhythmia detection, impending shock, and/or the like) and corresponding audible alerts in various languages.

When the suppression signal 404 causes the WMD 104 to withhold the audible alert in the first language, the application 310 uses the look up table 212 to retrieve the relevant audible alert in the second language. The retrieved audible alert in the second language is then played via the mobile device speaker 126, ensuring that the patient 306 hears the audible alert in the second language preferred by the patient 306. The application 310 may use a text-to-speech (TTS) technology to output the message or play a pre-recorded voice prompt. Steps 802-806 are already explained in detail in conjunction with the scenario 400 depicted in FIG. 4, therefore, for brevity, repeated description is omitted from FIG. 8.

FIG. 9 illustrates an example User Interface (UI) 900 of the mobile device 106 displaying language settings 902 of the mobile device 106, according to an embodiment of the present disclosure. FIG. 9 is explained in conjunction with FIGS. 1-8.

The UI 900 of the mobile device 106 allows the user 116 (e.g., a patient, a caretaker, an administrator, a healthcare provider, and/or the like) to select or adjust a second language (a user preferred language) in which the user 116 wants to receive audible alerts or voice prompts from the mobile device speaker 126 of the mobile device 106. The UI 900 is integral in ensuring that the user 116 may easily configure the medical system 100 to meet a language preference of the user 116, enabling effective communication and prompt understanding.

As illustrated in FIG. 9, the language settings 902 may include various sections. For example, the language settings 902 includes a preferred language section 904 with a dropdown menu or a selection menu 906 depicting different language options that the user 116 may choose or select as the second language. For example, the languages displayed under the selection menu 906 may include a range of options such as Language 1, Language 2, . . . , language N. Example of the languages including Language 1, Language 2, . . . , language N, may include, but are not limited to, English, French, German, Spanish, Chinese, and/or any other language that the medical system 100 may support. Each of the languages (e.g., Language 1 to Language N) may have corresponding buttons 908 (e.g., buttons 908a to 908n) for selection of the second language. The buttons 908a to 908n are collectively referenced to as buttons 908. The user 116 may select the second language from the range of options provided under the selection menu 906 by clicking on a corresponding button. Thus, to select a language, the user 116 needs to enable a corresponding button. For example, if the user 116 wants to select Language 4 as the preferred or second language, the user 116 is required to enable, press, or select a button 908d corresponding to language 4.

Accordingly, if a patient speaks language 4 (e.g., Spanish) as a primary language, the patient may select “language 4” from the dropdown or selection menu 906 by clicking on or enabling the button 908d corresponding to language 4. Once, the button 908d is enabled, an application hosted on the server 102 may be configured to mirror all audible alerts which are generated in a first language (e.g., English) by the WMD 104 in the selected “language 4.” The mirrored audible alerts in the second language (e.g., language 4) may be output by the mobile device speaker 126 of the mobile device 106.

Additionally, the language settings 902 may include a current language setting section 910, which provides the user 116 with an indication of a language currently in use for the audible alerts. The current language setting section 910 ensures that the user 116 is aware of the language in which the mobile device speaker 126 of the mobile device 106 may output the audible alerts. Referring back to the previous example, as the selected language is set to language 4, the current language setting section 910 may display language 4 (e.g., Spanish) as the language currently in use for outputting the audible alerts.

Further, the language settings 902 may include an automatic language sync section 912 with a button or toggle switch 914, which, when enabled or in an “ON” position, allows the application hosted on the server 102 to automatically use a default language or detect and match with a language of operating system of the mobile device 106. For example, if the mobile device 106 is set to language 2 (e.g., French), the application may automatically select language 2 as the second language or the user preferred language for mirroring the audible alerts, ensuring seamless integration with preferences of users. In such a case, the audible alert issued by the WMD 104 in English (e.g., “Battery is critically low. Please charge immediately”) may be mirrored by the application in French (e.g., “La batterie est presque vide. Veuillez charger immédiatement”).

Further, the language settings 902 may include a voice format section 916, allowing the user 116 to choose between different voice formats for the audible alerts. The voice format section 916 may include options such as different voices 918 (e.g., voices of male or female), different accents 920, or even voice speeds 922 (e.g., slow, normal, fast, and/or the like) and corresponding buttons for selecting the voice formats. For example, a patient may prefer to hear the mirrored audible alert in a female voice rather than a male voice, or at a slower pace if the patient has difficulty in processing fast speech. The section of the voice formats provides additional customization to language mirroring functionality.

In some implementations, the language settings 902 may include a preview section 924 that allows the user 116 to test language settings by playing a sample audible alert, which ensures that the user 116 may confirm the language and the voice format selections before proceeding with full setup of the mobile device 106. For example, when the user 116 clicks on a speaker icon 926 indicated in the preview section 924, the sample audible alert such as “Please replace the battery,” played in the selected language or the second language, thereby providing the user 116 an opportunity to hear how the mobile device 106 may sound during actual rendering of the audible alert.

The language settings 902 may include a save button 928 to finalize the selections. The user 116 may click on the save button 928 to save all the selections. Once the selections are saved, the application hosted on the server 102 may begin mirroring the audible alerts from the WMD 104 in the selected language and/or voice format. Such selections offer immediate reinforcement for audible alerts such as impending shock alerts, device malfunctions, low battery notifications, and/or the like. The above-described customization of the languages or the voice formats allows the medical system 100 to cater to diverse user needs, thereby improving clarity and understanding of critical health-related information. It may be noted that the medical system 100 supports a wide range of languages, accommodates different voice formats, and offers customization features to ensure that the user 116 receives clear and understandable alerts in the preferred languages, to improve patient compliance and response to alerts from the WMD 104.

Therefore, the implementations of the present disclosure, as described via the medical system 100 and exemplary scenarios, provide enhancement of interactions between the WMD or WCD 104 and the mobile application 310 installed on the mobile device 106. The medical system 100 addresses a critical need for effective communication of audible alerts, ensuring that users 116 (e.g., patients, bystanders, caregivers, and/or the like) receive audible alerts in a language the users understand and prefer.

In some embodiments, the medical system 100 may use an approach that not only issues the audible alerts but also provides text-based alerts in a users' preferred language, referred to as the second language. The text-based alerts improve accessibility by enabling the users 116 to read the alerts in addition to hearing the alerts. In some other implementations, the medical system 100 uses an approach where the mobile device 106 may be paired with hearing assistive devices (e.g., hearing aids, cochlear implants, and/or the like), and the audible alerts may be provided to the users 116 via the hearing assistive devices. Such approaches are particularly beneficial for users with hearing impairments, and the text-based alerts are also beneficial for those who prefer reading over listening. In some implementations, the text-based alerts may be provided to any of the users 116 (e.g., the caregiver, physicians, and/or the like). It may be noted that examples of the users 116 are already provided in some embodiments of the disclosure. To further aid in accessibility, the medical system 100 also provides haptic feedback, signaling to the users when the text-based alerts are being displayed. Thus, the dual modality, combining both the audible and text-based alerts, increases the likelihood that the users understand the alerts and take appropriate actions. In some cases, the text-based alerts in the second language also include an original audible alert in the first language, providing a comparative view for the users. This dual-language presentation ensures that the alerts are received clearly in both auditory and visual formats, reinforcing critical information for the users.

In some embodiments, instead of delivering the alerts as audible alerts, the medical system 100 provides the alerts purely as text-based alerts in the second language. In such embodiments, the text-based alerts are accompanied by a haptic notification to inform the users regarding the presence of the alerts. The text-based alerts accompanied by the haptic notification are useful when the audio alerts are not ideal, such as in noisy environments or when the users prefer visual communication. In some other embodiments, when the medical system 100 detects that an audible alert is required, the medical system 100 outputs the audible alert in the second language immediately following the original audible alert in the first language, ensuring that the users are provided with necessary alerts in the users' preferred language in a timely manner. In some embodiments, the medical system 100 includes a functionality of providing an alert (e.g., the audible alert, the text-based alert, the prompt, a notification, a haptic notification, and/or the like) to guide the bystanders or caregivers in emergency situations through a display, a speaker, and/or any other output unit (e.g., an indication through LED) of at least one of the WCD 104, the mobile device 106, the hearing assistive devices, and/or the like. For example, if the WCD 104 detects that the patient 306 is unconscious and experiencing an arrhythmia that requires Cardiopulmonary Resuscitation (CPR), the alert may be transmitted to nearby individuals of the patient 306 like the caregiver or the bystander, instructing them to contact emergency services and provide the CPR. The contact information of the emergency services may vary depending on the geographical region. For example, calling “911” to contact emergency medical personnel in the U.S. The transmission of the alert ensures that immediate action is taken while waiting for emergency medical personnel to arrive, increasing the chances of survival for the patient 306.

In some implementations, the mobile device 106 may be configured to listen for speech from nearby individuals (e.g., the bystanders, the caregivers, and/or the like) of the patient 306 and recognize a language being spoken by the nearby individuals before issuance of the audible alerts from the WCD 104 that are usually directed to the nearby individuals. For example, in scenarios when the WCD 104 is preparing to shock the patient 306, the audible alerts may be directed to the nearby individuals to not touch the patient 306, or to perform CPR if the defibrillation shocks fail to convert the rhythm. The mobile device 106 may then determine if the language being spoken by the nearby individuals is different from the selected language or the default language, and the mobile device 106 accordingly may provide the audible alert in the recognized language of the nearby individuals. For example, if the mobile device 106 is set to issue the audible alerts in English, but the nearby individuals are speaking Spanish, the mobile device 106 may detect the Spanish language and repeat the audible alert in Spanish, ensuring that the nearby individuals receive the instructions in a language they understand. S.

The medical system 100 also supports real-time streaming of data from the WCD 104 to the mobile application 310 or the language mirroring engine 114. The streaming includes information about the specific audible alerts that are about to be issued or have already been issued by the WCD 104. The mobile application 310 or the language mirroring engine 114 utilizes the information to provide immediate feedback to the users and ensures that the audible alerts in the second language are issued promptly after the WCD 104 finishes issuing the audible alert in the first language. Additionally, the WCD 104 transmits a message to the mobile application 310 or the language mirroring engine 114 indicating an event that triggered the audible alert, such as detection of an arrhythmia or a low battery alert. Thus, responsiveness of the medical system 100 is enhanced, enabling the mobile application 310 or the language mirroring engine 114 to accurately reflect underlying medical condition that triggered the audible alert, providing the users with more context for the action required.

The medical system 100 further provides an ability for the mobile application 310 or the language mirroring engine 114 to request a language setting of the WCD 104 or receive the language setting as a part of data stream along with the audio output state. The ability allows the mobile application 310 or the language mirroring engine 114 to confirm that the mirrored audible alerts in the second language are issued, ensuring that the audible alerts correspond to the users' preferred language. For example, if the mobile application 310 or the language mirroring engine 114 detects that the mobile device 106 is set to the second language, the mobile application 310 or the language mirroring engine 114 confirms that the mirrored audible alerts in the second language are issued in the second language, thus avoiding confusion or miscommunication.

In some embodiments, the mobile application 310 or the language mirroring engine 114 provides both the audible alerts in the first language and the second language sequentially. After outputting the audible alerts in the second language, the mobile application 310 or the language mirroring engine 114 immediately delivers the same audible alert in the first language. The dual-language output reinforces the audible alerts, making sure that the users receive necessary medical information in both the languages, providing confirmation and clarification when required. The dual-language output is beneficial when the users require extra confirmation of the audible alerts or when the users do not have understood the initial audible alert fully.

The medical system 100 supports various WMDs such as WCDs, Wearable ECG devices or any other medical device capable of generating audible alerts based on detected conditions. The language mirroring functionality may be applied to any WMD that outputs the audible alerts, making the medical system 100 highly versatile and applicable to a wide range of wearable devices, thus expanding the overall utility of the medical system 100. Additionally, the medical system 100 supports flexibility in a manner in which features are applied. For example, the features described in the present disclosure may be applied in different orders than described, individual features may be extracted and combined with others, or new features may be introduced, all while maintaining the described advantages of the medical system 100. As a result, the medical system 100 may be customized to meet the needs of different users, health conditions, and scenarios, making the medical system 100 a highly valuable tool in improving user compliance and health management.

In conclusion, the medical system 100 offers a comprehensive solution to improve interaction of users (e.g., the patients, bystanders, caregivers, and/or the like) with the WMDs by ensuring that the audible alerts are understood in the users' preferred language. The integration of the mobile application, language mirroring functionality, and flexible communication options (including voice, text, and/or haptic feedback) ensures that the medical system 100 is both accessible and user-friendly. Thus, the medical system 100 enhances the communication, facilitates understanding, and promotes timely and accurate responses to the audible alerts, improving user health management and compliance. Accordingly, timely and accurate responses to alerts or prompts play a critical role in treating severe cardiac conditions and can potentially save users' lives in life-threatening situations, such as sudden cardiac arrest.

Other embodiments include combinations and sub-combinations of features described or shown in the drawings herein, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A medical system comprising:
a Wearable Medical Device (WMD) comprising at least one medical device speaker, wherein the WMD is configured to provide therapy to a patient upon detection of a cardiac condition of the patient based on patient data; and
a mobile device comprising at least one processor and at least one mobile device speaker, wherein the mobile device is in communication with the WMD, wherein the at least one processor is configured to:
receive an indication of an audible alert to be output by the at least one medical device speaker of the WMD in a first language; and
cause the at least one mobile device speaker to output the audible alert in a second language.

2. The medical system of claim 1, wherein the indication is the audible alert in the first language output by the at least one medical device speaker.

3. The medical system of claim 1, wherein the indication is a pre-alert signal.

4. The medical system of claim 3, wherein the pre-alert signal is output by the at least one medical device speaker, and wherein the pre-alert signal has a frequency and/or amplitude that is inaudible to humans.

5. The medical system of claim 3, wherein the pre-alert signal is an electronic communication transmitted by the WMD to the at least one processor of the mobile device.

6. The medical system of claim 3, wherein the at least one processor is further configured to send a suppression communication to the WMD, in response to receiving the pre-alert signal, and wherein the suppression communication is operable to override the audible alert in the first language with the audible alert in the second language.

7. The medical system of claim 1, wherein the at least one processor is further configured to display a visual alert in the second language corresponding to the audible alert on a display of the mobile device.

8. The medical system of claim 1, wherein the audible alert comprises an alert associated with WMD conditions and/or associated with patient data.

9. The medical system of claim 1, wherein the at least one processor is further configured to detect a language of a bystander, and cause the at least one mobile device speaker to output the audible alert in the detected language.

10. A method for outputting an audible alert in a second language, the method comprising:
transmitting, via a Wearable Medical Device (WMD), an indication of the audible alert to be output by at least one medical device speaker of the WMD in a first language;
receiving, at a mobile device, the indication; and
outputting, via at least one mobile device speaker, the audible alert in the second language, wherein the WMD is configured to provide therapy to a patient upon detection of a cardiac condition of the patient based on patient data.

11. The method of claim 10, wherein the indication is the audible alert output by the at least one medical device speaker.

12. The method of claim 10, wherein the indication is a pre-alert signal.

13. The method of claim 12, wherein the pre-alert signal is output by the at least one medical device speaker, and wherein the pre-alert signal has a frequency and/or amplitude inaudible to humans.

14. The method of claim 12, wherein the pre-alert signal is an electronic communication transmitted by the WMD to the mobile device.

15. The method of claim 14, further comprising:
in response to receiving the pre-alert signal, transmitting, via the mobile device, a suppression signal to the WMD; and
overriding output of the audible alert in the first language with the second language.

16. The method of claim 10, further comprising displaying, on a display of the mobile device, a visual alert in the second language corresponding to the audible alert.

17. The method of claim 10, wherein the audible alert comprises alerts associated with WMD conditions and/or alerts associated with patient data.

18. The method of claim 10, wherein the second language is selected by the patient and/or based on a language setting of the mobile device.

19. A non-transitory computer readable medium, encoded with instructions stored thereon for outputting an audible alert in a second language, that when executed by at least one computing device causes the at least one computing device to perform operations for outputting the audible alert in the second language, the operations comprising:
transmitting, via a Wearable Medical Device (WMD), an indication of the audible alert to be output by at least one medical device speaker of the WMD in a first language;
receiving, at a mobile device, the indication; and
outputting, via at least one mobile device speaker, the audible alert in the second language, wherein the WMD is configured to provide therapy to a patient upon detection of a cardiac condition of the patient based on patient data.

20. The non-transitory computer readable medium of claim 19, wherein the indication is a pre-alert signal or the audible alert output by the at least one medical device speaker of the WMD.
